(19) United States
(12) Patent Application Publication
(10) Pub. No.: US 2009/0074697 A1
(43) Pub. Date: Mar. 19, 2009

(54) Use of monosaccharides and disaccharides as complete replacements for synthetic fixative and styling polymers in hair styling products

(75) Inventor: Angelika F. Huynh, Walnut, CA (US)

Correspondence Address: HENKEL CORPORATION, 1001 TROUT BROOK CROSSING, ROCKY HILL, CT, 06067, US

(73) Assignee: Henkel AG & Co. KGaA

(21) Appl. No.: 12/283,985

(22) Filed: Sep. 16, 2008

Related U.S. Application Data
(60) Provisional application No. 60/994,331, filed on Sep. 18, 2007.

(51) Int. Cl. A61K 8/73; A61Q 5/06

(52) U.S. Cl. 424/70.13

(57) ABSTRACT

New hair styling compositions are described that use monosaccharides and/or disaccharides instead of synthetic fixative polymers or copolymers to provide excellent hold to hair without flaking.

### PRIORITY APPLICATION

This application claims the benefit of and priority to U.S. Provisional Patent Application No. 60/994,331, filed Sep. 18, 2007 and entitled “USE OF MONOSACCHARIDES AND DISACCHARIDES AS COMPLETE REPLACEMENTS FOR SYNTHETIC FIXATIVE AND STYLING POLYMERS IN HAIR STYLING PRODUCTS”, which is incorporated by reference herein in its entirety.

### FIELD OF INVENTION

The present invention relates to hair styling/hold compositions and specifically to hair styling/hold compositions comprising monosaccharides and/or disaccharides as complete replacements for synthetic hair fixative polymers and copolymers.

### BACKGROUND

It is very desirable in personal grooming to maintain one's hair in a particular setting, and a common method of accomplishing this is to apply a hair styling composition to the hair and allowing it to dry and set either on its own or with heat from a hair dryer or hot curlers. Hair setting compositions can assist in styling the hair and they provide a temporary hold to the hair while imparting appearance benefits such as shine. Hair styling products may come in various forms, including non-aerosol or aerosol sprays, gels, foams and lacquers. The objective in formulating hair styling products has always been to optimize a number of interrelated physical properties of the product, seen both before and after drying. These important parameters include achieving a desirable viscosity for the product so that it dispenses properly and predictably. If manually applied into the hair by hand, it is important to optimize the feel of the styling product prior to and during the spreading into the hair. Also important is to optimize final hold when dry, the ability to easily wash the product out with shampoo, and to minimize flaking. These parameters are evaluated by various visual and tactile sensory methods including viewing, touching, combing and brushing. Important parameters to optimize include shine, cleanliness, grease return, stiffness, tackiness, bounce, ability to re-style, combing ease, residue/flaking during comb/brush-through, static electricity, smoothness, and the like.

Compositions usable for hair styling products are well known in the art. Most commercial hair styling compositions include synthetically derived hair fixative and styling polymers, modified starches, natural gums and resins, cellulosic or synthetic polymer thickeners, polymer film modifiers, solvents, co-solvents, colors, fragrances, preservatives and the like. Synthetic styling and fixative polymers typically include nonionic, anionic or cationic fixative polymers and copolymers, which operate as film-formers. Some commercially available polymers that have been used for some time now in various combinations include; polyvinylpyrrolidone (PVP) sold under the trade name of Luviskol-PVP® from BASF, or PVP from ISP; polyvinylpyrrolidone/vinylacetate copolymer (PVP/VA) sold under the trade name PVP/VA from ISP; vinyl pyrrolidone/dimethylaminoethyl methacrylate copolymer sold under the trade name of Styleze® CC-10 from ISP; vinyl acetate/butyl maleate/isobornyl acrylate copolymer sold under the trade name Advantage® Plus from ISP; vinyl caprolactam/vinyl pyrrolidone/dimethylaminoethyl methacrylate copolymer sold under the trade name Advantage® S from ISP; polyquaternium-69 sold under the trade name Aquastyle® 300 from ISP; polyquaternium-11 (quaternized vinyl pyrrolidone/dimethylaminoethyl methacrylate copolymer) sold under the trade name Gafquat®-755N and Gafquat® 440 from ISP; and, polyquaternium-28 (vinylpyrrolidone/methacrylamidopropyl trimethyl ammonium chloride copolymer) sold under the trade name Gafquat® HS-100 from ISP. These synthetic materials are also referred to as “fixative resins”, “fixative copolymers”, “fixative polymers”, “fixatives”, “resins”, or “film-formers”.

When a hair setting composition is dried on the hair, it is important to optimize strength and elasticity of the film on the hair, and to ensure the dried film on each hair shaft does not flake or dust when the hair is subject to stress throughout the day, such as combing. Additionally, the set hair must be tack-free, clear, glossy, and humidity-resistant, yet still be easily washed out of the hair with shampoo products. To achieve these desirable attributes, it is common to blend both synthetic copolymers of the type mentioned above with a film-forming starch-based material such as modified corn starch sold under the trade name Amaze® from National Starch/Akzo Nobel. Such combinations of starches or starch derivatives with synthetic vinyl copolymers are thoroughly mentioned in U.S. Pat. No. 6,413,505 (Vitale et al.), incorporated herein in its entirety by reference.

When hair styling fixative polymers and starch-based materials do not provide the desired rheology to the product, formulators use rheology modifiers or gellants in addition to the film former polymers. These ingredients may include other synthetic polymers, gums, emulsifiers, waxes, and the like, to achieve the target rheology for the product. Some of the simplest thickeners used are homopolymers such as high molecular weight polyacrylates. Other common ingredients that help optimize the physical properties of hair hold compositions include petrolatum and various fatty alcohols.

Most “hard-hold” hair styling products on the market today rely on high concentrations of synthetic fixative polymers and copolymers to provide a desirous stiff feel and a high level of hold. Primarily for cost reasons, formulators have tried to reduce the amount of synthetic resins in hair-hold compositions by using saccharidic substances, including oligosaccharides and polysaccharide materials including the modified starches as mentioned above. However, it is well known in the industry that addition of simple sugars to hair hold compositions can lead to tacky formulas and increased flakiness during comb-through. In fact, it is well known that there is an upper limit to how much sugar can be added to hair-hold compositions to improve tactile and elastic properties, enhanced moisture retention, regeneration of split-ends, added strength, and the like, without causing deleterious properties. Indeed, the addition of sugars to hair styling compositions has been mostly to achieve some of these performance benefits mentioned above rather than as a strategy for reducing the amount of synthetic fixative polymer in the composition as is done with starch derivatives.

For example, U.K. Patent Application Publication GB2134784A (Grollier, et. al) describes combinations of anionic and cationic polymers with sugars and a salt to form a hair conditioner. These inventors claim an improvement to the rheological characteristics of the product, better bulk and hold, along with an increase in time before the hair becomes greasy again. The sugars described for use include maltose, mannose, lactose, levulose, raffinose, glucose and fructose. However, the compositions still contain percentages of synthetic cationic or cationic-behaving polymer (such as vinyl pyrrolidone/dialkylaminoalkyl acrylate or methacrylate co-polymer, or a cellulose ether derivative with a quaternized ammonium group, or cationic polysaccharide, or the like), and anionic polymer (such as acrylic acid or sulfonic acid derived materials) together with the sugar. In other words, complete replacement of synthetic fixative polymers with one or more saccharides is not claimed or suggested as even possible.

U.S. Pat. No. 5,833,968 (Keil, et. al) describes sprayable hair styling gels comprising disaccharide or mixtures of disaccharide and oligosaccharide (3-6 monosaccharide units), along with cationic polymer and a thickener (preferably polyacrylic acid), wherein the compositions are devoid of low molecular weight alcohols and devoid of metal salts (perhaps in view of '748 Grollier, et. al, supra). This invention achieved shear-thinning gels that disperse more uniformly onto the hair, although these compositions still contain hair fixative polymers.

U.S. Pat. No. 6,800,302 (Cannell, et. al) describes compositions that incorporate C₁-C₅ monosaccharides that are substituted with a C₁-C₂₂ carbon chain. These compositions may also include ordinary C₃-C₇ sugars for the purposes of aiding moisture retention. However, the compositions described by Cannell still include at least one styling or fixative polymer and there is no mention of the possibility for ordinary sugars to substitute for styling and fixative polymers.

U.S. Pat. No. 7,179,451 (Brandt, et. al) describes the addition of oligosaccharide or polysaccharide to traditional hair-hold compositions containing resins as a way to reduce the high concentrations of vinyl and acrylic polymers in the formulas and to maintain excellent stiffness, improve curl retention and minimize flaking. Brandt claims only the use of oligosaccharides and polysaccharides (i.e., saccharides with greater than two sugar monomer units) in their formulas. Most importantly, Brandt teaches that curl retention ability “does not occur with saccharides having two or less monomer units” (i.e., monosaccharides or disaccharides), and that “when disaccharides are used, a great deal of stickiness or unnatural feel results”, ('451, Column 3, Lines 36-45 of the '451 patent).

Lastly, published U.S. Patent Application Publication No. 2003/0021758 (Cannell, et. al, now abandoned) describes heat-activated styling compositions minimally comprising a C₃ to C₅ monosaccharide and a “film-forming agent”. The examples include use of polyquaternium-16, 46, and 44 and polyvinylopyrrolidone-modified starch as the film former, along with xylose as the preferred monosaccharide. The use of disaccharides is not disclosed in this published application and even with the use of xylose there is still reliance on a synthetic film forming polymer, minimally polyvinyl pyrrolidone.

Thus what is needed in the industry, and what is entirely lacking in the art today, are hair styling compositions entirely devoid of polymeric film forming polymers and copolymers (i.e., fixatives), which still provide consumer acceptable feel, hold and shine without undue flaking.

### SUMMARY OF THE INVENTION

The present invention provides hair styling compositions that give remarkable hard-hold and reduced flaking characteristics even though the compositions are entirely devoid of synthetic polymer and copolymer film forming resins. These new compositions incorporate monosaccharides and/or disaccharides as the complete substitute for the synthetic resin. This not only has been unachieved in the past, but the prior art teaches against the replacement of all the resin with any mono- or disaccharide since it was believed that such a strategy will not lead to a usable composition. The present invention provides compositions utilizing starch-based or cellulose-based materials, along with mono- and/or disaccharides, to circumvent the need for synthetic fixatives.

Not being bound by any theory, mono- or disaccharides may interact with the starch-based or cellulose-based ingredients on the shafts of the hair. However, the choice of using a particular monosaccharide, disaccharide, or mixtures of both in the compositions of the present invention, remains somewhat empirical.

The compositions of the present invention minimally comprise; (a) at least one monosaccharide or disaccharide or combinations of the two; (b) a modified starch or modified cellulose; and, (c) water, wherein the composition is devoid of synthetic fixative polymers or copolymers. The monosaccharides and disaccharides that find use in the present invention may be naturally occurring (including and preferably the simple “sugars”) or synthetically derived, and include a great number of small organic substances.

### DETAILED DESCRIPTION OF THE INVENTION

The following description is of exemplary embodiments only and is not intended to limit the scope, applicability or configuration of the invention in any way. Rather, the following description provides a convenient roadmap for implementing exemplary embodiments of the invention. Various changes to the described embodiments may be made in the function and relative amounts of the ingredients described without departing from the scope of the invention as set forth. Additionally, though demonstrated herein in terms of hair styling/setting thick-liquid to paste/gel compositions, other product forms the present invention may take include thin pourable liquids, aerosol hair sprays, spritzes, foams, crèmes, pastes, non-runny gels, mousses, pomades, lacquers, non-aerosol pump-spray liquids and the like. Furthermore, the compositions of the present invention may include various adjuvant such as deodorants, fragrances, aromatherapy essences, herbs, infusions, antimicrobials, pediculicides, stimulants, essential oils, hair coloring, dying or tinting agents, anti-gray agents, sun-blockers, vitamins, antioxidants, surfactants and other wetting agents, Rogaine®, mica, silica, metal flakes or other glitter-effect materials, conditioning agents, anti-static agents, opacifiers, detackifying agents, penetrants, preservatives, and emollients and the like, and these compositions having additional consumer, cost, performance, and/or stability benefits (perceivable or otherwise) likewise fall within the ambit of the present invention.

That being said, the present invention provides compositions useful for styling and setting hair that comprise monosaccharides, disaccharides or mixtures of the two saccharides as the principle styling agent in the composition. Most remarkable, these compositions are entirely synthetic fixative free, yet they still impart firm styling when dried on the hair. Thus, the compositions of the present invention minimally comprise;

(a) at least one monosaccharide or disaccharide, or combinations of the two;

(b) a modified starch or modified cellulose, or combinations of the two; and,

(c) water, wherein the composition is devoid of synthetic fixative polymers or copolymers.

Additionally, the compositions of the present invention may optionally include rheology modifying polymers, an emulsified oil phase with, for example, petrolatum, fatty alcohols, diols and polyols, emulsifiers, humectants, silicone derivatives, proteins, conditioners, chelants, solvents, and combinations of the adjuvants mentioned previously to impart consumer perceivable benefits, cost/manufacturing benefits, and/or to adapt the base formulations comprising the essential ingredients, ((a) to (c) above), to a particular physical product form, a specific delivery method, and/or a preferred package.

That being said, the compositions of the present invention include a monosaccharide or a disaccharide, or combinations of the two types. These are molecules having one or two monomer units respectively. For simplicity, these materials may also be referred to here as “sugars”, consistent with the common naming of these lower molecular weight substances and to avoid confusion with “saccharides”, the generic term sometimes given to the polymeric materials such as starches that the present invention includes. Thus herein, “starch”, “starch derivatives”, “cellulose”, and “cellulose derivatives”, rather than “polysaccharide” or “saccharine polymer” will be used to indicate the high molecular weight materials based on carbohydrate backbones. Additionally, distinction is made between mono- and disaccharides in the present invention and the “oligosaccharides” that appear in some prior art. Oligosaccharides are molecules with greater than two monomer units, which fall outside the scope of the present invention. Of importance to the scope of the present invention is that the compositions may contain synthetic or naturally occurring mono- and/or disaccharides, although the naturally occurring substances are preferred for the sake of reduced cost and the ability to make a marketing claim of “natural” hair care product.

Sugars that find use in the hair hold compositions of the present invention include monosaccharides having three to seven carbons (C₃ to C₇), with these chosen from the general classes of ketose or aldose. Most preferred is to use a monosaccharide having a five or six carbon chain (C₅ or C₆ sugars). As mentioned previously, any sugar for use in the present invention may be found in nature or synthetically obtained, and this not only includes structural variants (for example, a sugar with structure unknown in nature, or synthetic structural isomers of known sugars, such as varying the position of the carbonyl group in a ketose), but also the D-form, L-form, or mixtures therein (i.e., the stereoisomers of natural sugars, or optically active materials produced through asymmetric synthesis, or racemic materials produced in the absence of asymmetric processes). The C₃ to C₅ monosaccharides may be chosen from any triose, tetrose and pentose. Non-limiting examples of C₃ to C₅ monosaccharides include aldopentoses (such as xylose, arabinose, lyxose, and ribose), ketopentoses (such as ribulose and xylulose), aldotetroses (such as erythrose and treose), ketotetroses (such as erythrulose), aldotrioses (such as glyceraldehyde) and ketotrioses (such as dihydroxyacetone). Non-limiting examples of C₆ to C₇ sugars include the hexoses (for example, allose, altrose, glucose, mannose, gulose, idose, galactose, talose, sorbose, psicose, fructose, and tagatose); and the heptoses (for example sedoheptulose). As mentioned, the ketoses may be synthetic in which case the keto (carbonyl) group does not need to be on the second carbon of the chain. Additionally, synthetic monosaccharides may be used that have additional degrees of unsaturation, for example where one of the hydroxyl groups may be oxidized to a keto group, or a substance where one or more —OH group(s) is/are gone (i.e., particular “deoxy-”compounds rather than true carbohydrates). In general, the preferred monomeric sugars that find use in the hair hold compositions of the present invention may contain 5- or 6-carbon atoms in their chains and an oxygen substituent at each carbon, that oxygen being either a hydroxyl (reduced) group or carbonyl (oxidized) group. The most preferred monosaccharides for use in the present invention include naturally occurring carbohydrates of the general formula Cₓ(H₂O)ₓ, where x is 5 or 6. It should be recognized that single monosaccharides may be used or combinations of two or more monosaccharides mixed together in the same hair styling composition. Preferably the monosaccharide(s) is/are incorporated into the hair styling compositions of the present invention at from about 0.1% to about 50% by weight of the total composition.

Disaccharides that find use in the hair hold compositions of the present invention include covalently linked combinations of any two of the above-mentioned monomer materials, including dimerization of two identical monomers or linkage of any two dissimilar monomers, both at any structural position on the monomers. These disaccharides may be naturally occurring or synthetically derived. Most preferred for use in the present invention are the natural disaccharides such as maltose, sucrose, cellobiose, trehalose and lactose, again for cost and for the opportunity to make “natural” marketing claims. It should be recognized that although a disaccharide may be added to the present compositions, it might react in situ to the mixture of monosaccharides. For example, although sucrose may be added to a preferred composition herein, it may cleave to “invert sugar” (an equimolar mixture of glucose and fructose) in situ, either immediately or over the course of storage and merchandizing. Preferably the disaccharide(s) is/are incorporated into the hair styling compositions of the present invention at from about 0.1% to about 50% by weight of the total composition

Most preferred for use in the present compositions is maltose, fructose, glucose or sucrose, or combinations of any of these mono and disaccharides, at a total amount of from about 0.1% to about 50% by weight in the total composition. More preferred is to use maltose, fructose, glucose or sucrose, or combinations thereof, at a level of from about 1% to about 30% by weight in the total composition.

The compositions of the present invention also include starch, modified starches, cellulose, or cellulose derivatives, or combinations of these, for combining with the mono- and/or disaccharide to obtain a quality dried film on the hair and for adjusting the viscosity of the actual composition. For example, of use in the compositions of the present invention are celluloses, cellulose derivatives, cellulose gums, ethoxylated celluloses, starch or gums, guar gum, guar hydroxypropyl trimonium chloride, xanthan gum, karaya gum, and the like, and combinations thereof. Also of use in the present invention are pregelatinized crosslinked starch derivatives, including hydroxypropyl starch phosphate, as described in U.S. Patent Application Publication No. 2005/0191264 (Detert, et. al) and available from National Starch under the trade name, Structure XL®. Preferred cellulosic materials for use in the compositions of the present invention include nonionic or cationic modified cellulose such as cellulose ethers including methyl cellulose, carboxymethyl cellulose, hydroxypropylmethyl cellulose, hydroxyethyl cellulose such as UCARE® Cellosize® PCG-10 from Americhol, hydroxypropyl cellulose, and ethyl hydroxyethyl cellulose, Kitamer® PC, a chitosan carboxylate and Kytamer® L, a chitosan lactate obtained from Amerchol, sodium alginate, agarose, amylopectins, amyloses, arabinans, arabinogalactans, arabinoxylans, carrageenans, gum arabic, additional cellulose derivatives such as carboxymethyl guar gum, carboxymethyl-(hydroxypropyl)guar gum, hydroxyethylguar gum, hydroxypropylguar gum, cationic guar gum, chondroitins, chitins, chitosans, cocodimonium hydroxypropyl oxyethyl cellulose, colominic acid [poly(N-acetyl-neuraminic acid], corn starch, potato starch, oxidized or modified corn starch such as Amaze® 28-1890 from National Starch, curdlan, dermatin sulfate, furcellarans, dextrans, cross-linked dextrans known as dextranomer (such as Debrisan® from Pfizer), dextrin, emulsan, flaxseed saccharide (acidic), galactoglucomannans, galactomannans, glucomannans, glycogens, guar gum, or hydroxyethylstarch, hydroxypropylstarch, hydroxypropylated guar gums, gellan gum, glucomannans, gellan, gum ghatti, gum karaya, gum tragacanth (tragacanthin), heparin, hyaluronic acid, inulin, keratan sulfate, konjac mannans, laminarans, laurdimonium hydroxypropyl oxyethyl cellulose, liposan, locust bean gum, nigeran, nonoxylnyl hydroxyethyl cellulose, okra gum, pectic acids, pectins, polydextrose, protopectins, psyllium seed gum, pullulan, sodium hyaluronate, steardimonium hydroxyethyl cellulose, raffinose, rhamsan, tapioca starch, welan, levan, scleroglucan, stachyose, succinoglycan, wheat starch, xanthan gum, xylans, xyloglucans, and mixtures thereof. Microbial polysaccharides can be found in the fourth edition of Kirk-Othmer Encyclopedia of Chemical Technology, Fourth Edition. Vol. 16, John Wiley and Sons, NY pp. 578-611, 1994. Complex carbohydrates can be found in the fourth edition of Kirk-Othmer Encyclopedia of Chemical Technology, Fourth Edition. Vol. 4, John Wiley and Sons, NY pp. 930-948, 1994.

Of particular use in the present invention are hydroxyethylcellulose and a modified cornstarch, used individually or in combination. Most preferred for use in the compositions of the present invention is from about 0.01% to about 20% total starch/cellulose materials by weight in the composition. Most preferred is to use from about 0.01 % to about 20% of either hydroxyethylcellulose (e.g. the Cellosize® products from Americhol), or hydroxypropyl starch phosphate (e.g. the Structure® products from National Starch/Akzo Nobel), or Modified Corn Starch (e.g. the Amaze® starch products from National Starch/Akzo Nobel), or combinations of any of these in the same composition, totaling at from about 0.01% to about 20% by weight.

Optionally, the compositions of the present invention may include a diol or polyol. Polyols may function as humectants, which are discussed separately below. The polyols that find use in the present invention may be natural or synthetically derived, and may include straight chain (i.e., acyclic), cyclic, or combinations of straight chain and cyclic structures, including small molecular weight substances and up to polymeric materials. For example simple diols or more complex polyols that may find use in the present compositions include, but are not limited to, ethylene glycol, propylene glycol, dipropylene glycol, hexylene glycol, MP Diol® Glycol (methyl propylene diol), 1,4-butanediol, 1,5-pentanediol, 1,6-hexanediol, erythritol, glycerol (i.e., 1,2,3-propanetriol and also called glycerin or glycerine), diglycerine, various reduced sugars (sugar alcohols) such as mannitol, sorbitol and xylitol, isomalt, lacitol, maltitol, various HSH's (hydrogenated starch hydrosylates), polyethylene glycol, and polypropylene glycol, and the like. Polyols that contain, in addition to the requisite minimum (2) hydroxyl groups by definition, other functional groups in the molecule such as aldehydes, ketones, carboxylate, thiols, etc., are also of use in the present invention. For example, 1,6-dihydroxhexane-3-one may find use as the polyol in the present invention and is a good example of a molecule that is a polyol (a diol) but with additional functionality (a keto group). Other multifunctional polyols include non-limiting examples 2,5-dihydroxyhexal, 2,5-dihydroxyhexanoic acid, methyl 2,3,5-trihydroxyhexanoate, and the like. Many small organic molecules, or mixtures of them, may find use in the compositions of the present invention provided they minimally have two or more hydroxyl groups (i.e., are polyols). Polymeric polyols are also useful and the most preferred of these is polyethylene glycol having general formula H—(OCH₂CH₂)ₙ—OH where n is from about 4 to about 12. Most preferred for use in the compositions of the present invention include propylene glycol, ethylene glycol, glycerol, and polyethylene glycol. The polyol or polyols is/are preferably incorporated in the compositions of the present invention at from about 0.1% to about 5% by weight in the composition. Most preferred is to use a diol or polyol such as propylene glycol, polyethylene glycol (such as Carbowax® PEG-200 from Union Carbide), and/or glycerol, or mixtures of these at from about 0.1% to about 5% by weight in the composition.

The compositions of the present invention may also include a rheology-modifying polymer, such as a high molecular weight polyacrylate, co-polymer or cross-linked polymer. Such rheology-modifying polymers may simply thicken, or may give elastic or pseudo-elastic characteristics that may be necessary depending on the final form of the hair care product (e.g. a gel intended to be squeezed out from a plastic tube). Rheology modifiers can be utilized alone or in combination so long as the chosen materials are compatible with the hair styling composition. Rheology modifiers are also important when the compositions of the present invention are to be adapted to gels and pomades and the like, products that are intended to be rubbed between the palms of the hands and applied to the hair. Rheology-modifiers may include, but are not limited to, acrylic acid homopolymers and acrylates co-polymers under the Carbopol® trade name from Lubrizol, (for example, Carbopol® 934, 940, 941, 980, 981, 1342, 1382, 2984, 5984, EDT-2020, EDT-2050, ULTREZ® 10 Polymer, ULTREZ® 20 Polymer and ULTREZ® 21 Polymer), acrylates/Steareth-20 itaconate copolymer, acrylates/ceteth-20 itaconate copolymer from National Starch, Bentonite, PVM/MA Decadiene Crosspolymer from International Specialties Products, Acrylates/steareth-20 methacrylate copolymer, associative thickeners such as Acrysol® ICS-1, Acusol® 820, and Acusol® 830 from Rohm and Haas, acrylamide/sodium acrylate copolymer, Hostacerin® PN 73, Hoecsht AG., acrylate copolymer (Antil® 208) supplied by Degussa-Goldschmidt, acrylic acid/acrylonitrogens copolymer (Hypan® SA-100H, SR-150H) supplied by Lipo, Acrylic/acrylate copolymer (Carboset® 514, 515, 525, XL-19, XL-19X2, XI-28, XL40, 526) supplied by Noveon, Ammonium acrylateslacrylonitrogens copolymer (Hypan® SS-201) from Lipo, Quaternium-18 Bentonite, sodium salt of crosslinked poly(acrylic acid) under the tradenames PNC® 430, PNC® 410, PNC® 400 from 3V, Stearalkonium Bentonite, Claytone, supplied by Southern Clay, Quaternium-18 Hectorite (Bentone 38), Stearalkonium Hectorite (Bentone 27) supplied by Rheox, acrylamide/sodium acrylate copolymer (Hostacerin PN 73) supplied by Hoechst, Poly(acrylic acid) known as Carbopol® 400 series (Lubrizol) or Aquatreat® (Alco), polyquaternium-18 (Mirapol® AZ-1) from Rhone Poulenc, polyquaternium-27, polyquaternium-31, polyquaternium-37, trihydroxystearin (Thixcin from Rheox; Flowtone from Southern Clay), Dimethylaminoethyl methacrylamide and acrylamide copolymer (Salcare® SC63 from Ciba Specialties), and acrylic polymer cationic thickening agents (Synthalen® CR and its related compounds) from 3V Sigma. Other rheology-modifying polymers can be found in the “The Encyclopedia of Polymers and Thickeners for Cosmetics,” Cosmetics and Toiletries, Lochhead, R., pp. 95-138, Vol. 108, (May 1993). It should be understood that many of the acrylic acid polymers and co-polymers require neutralization to thicken and this can be accomplished in situ by the addition of any alkaline material to adjust the pH of the final composition, (e.g., addition of hydroxides, ammonia, amines, alkanolamines, and the like).

The preferred rheology-modifying polymer for use in the present compositions is Carbopol® ULTREZ® 10, Carbopol® ULTREZ® 20, Carbopol® ULTREZ® 21, Carbopol® 1342 and Carbopol® 1382 (all from Lubrizol), used individually or in various combinations, at a level of from about 0.01% to about 2% by weight of the total composition.

The compositions of the present invention may also include petrolatum. Petrolatum is a mixture of hydrocarbons that finds use in various personal care products. Preferred petrolatum includes white petrolatum USP, petrolatum USP, mineral jelly and ointment base. The melting point ranges of the preferred petrolatum for use in the present invention may be from about 80° F. to about 135° F. Most preferred is to use UltraPure® Liquid Petrolatum USP from Ultra Chemicals that has a melting point range of about 105°-115° F., or various grades (ranging in color) of Penreco® Petrolatum USP having melting point ranges around 122°-135° F. Preferred is to incorporate from about 0.01% to about 10% of petrolatum by weight in the composition.

Fatty alcohols that may find use in the compositions of the present invention include naturally derived and synthetic materials. These are high molecular weight straight or branched chain primary alcohols. Most preferred for use in the present invention include lauryl (C₁₂), myristyl (C₁₄), cetyl or palmityl (C₁₆), stearyl (C₁₈), oleyl (C₁₈-unsaturated) and linoleyl (C₁₈-polyunsaturated) alcohols, or cetearyl (blend of cetyl and stearyl alcohols, such as the 50/50 mixture available from RITA Corp.). Preferred is use cetyl alcohol or cetearyl alcohol. The fatty alcohol in the present invention may be incorporated at from about 0.01% to about 10% by weight in the composition.

Also of use as an optional ingredient in the compositions of the present invention include emulsifiers. Emulsifiers for use in cosmetic applications are amply listed in McCutcheon's Emulsifiers and Detergents. Many suitable emulsifiers are nonionic esters or ethers comprising a polyoxyalkylene moiety, (or less accurately named polyalkylene glycol moiety), especially a polyoxyethylene moiety, often containing from about 2 to 80, and especially 5 to 60 ethylene oxide (EO) units. The hydrophilic moiety can contain polyoxypropylene (PO) rather than polyoxyethylene (EO), or may be mixed hydrophilic alkoxylation (EO/PO). The emulsifiers additionally may contain a hydrophobic alkyl, alkenyl or arylalkyl moiety, normally containing from about 8 to 50 carbons. The hydrophobic moiety can be either linear or branched and is often saturated, though it can be unsaturated, partially or fully hydrogenated, and/or substituted with various groups such as hydroxyl or be fluorinated. The hydrophobic moiety can comprise a mixture of chain lengths, for example those derived from tallow, lard, palm, castor, canola, sunflower seed, or soya bean oil and the like. Such nonionic esters or ethers can also be derived from a polyol compound such as glycerol, sorbitol or other alditols.

Examples of useful emulsifiers for hair styling compositions include cetyl and steryl derivatives such as ceteareth-10 to -25, ceteth-10-25, steareth-10-25 (which are C₁₆ to C₁₈ alcohols ethoxylated with 10 to 25 ethylene oxide residues), cetyl lactate, caprilic/capric triglyceride, propylene glycol dicaprylate/dicaprate (e.g., Liponate® PC from Lipo), myristyl lactate, myristyl myristate, pentaerylthrityl tetraoleate, pentaerythrityl tetrastearate, isopropyl myristate, isopropyl palmiate, various cetyl esters (e.g., a mixture supplied by Lipo as Liponate® SPS), stearyl stearate, butyl stearate, myreth-3 myristate, pentaerythrityl tetrabehenate, diisopropyl adipate, dipentaerythrityl hexacaprylate/hexacaprate, neopenyl glycol dicaprylate/dicaprate, tridecylsearate, tridecyl trimellitate, PEG-4 diheptanoate, pentaerythrityl tetracaprylate/tetracaprate, isocetyl stearate, ethylhexyl palmitate, C₁₂-C₁₅ alkyl benzoate (e.g., Liponate® NEB from Lipo), cetyl ricinoleate, isostearic acid, PEG-5 to PEG-40 stearate, PEG-5 to PEG-40 distearate and PEG-5 to PEG-40 tallow, lard, palm, castor, canola (tribehenin), hydrogenated castor, sunflower seed, or soya bean oil. Other suitable examples include C₁₀-C₂₀ fatty acid mono, di or tri-glycerides. Further examples include C₁₈-C₂₂ fatty alcohol ethers of polyethylene oxides (8 to 12 EO). Other examples of useful emulsifiers are fatty acid mono or diesters of polyhydric alcohols such as glycerol, sorbitol, erythritol or trimethylolpropane. The fatty acyl moiety may be from C₁₄ to C₂₂ and optionally saturated, including cetyl, stearyl, arachidyl and behenyl. Examples include monoglycerides of palmitic or stearic acid, sorbitol mono or diesters of myristic, palmitic or stearic acid, and trimethylolpropane monoesters of stearic acid. Additional glycerides include polyoxyethylene fatty acid glyceride esters, most preferably polyoxyethylene caprylic/capric mixed glyceride esters including PEG-6 caprylic/capric glyceride (Glycerox® 767 from Croda).

Other preferred emulsifiers include any combination of fatty alcohols (such as mentioned previously), phosphate-based emulsifying waxes, sorbitan monooleates and stearates and other carbohydrate esters of fatty alcohols and their ethoxylated derivatives, and the polyalkylene glycols and polyethoxylated waxes. For example, a preferred fatty alcohol blend for optional use in the present invention is Crodafos® CES (white solid or flakes) from Croda, which is a blend of ceteryl alcohol, dicetyl phosphate and ceteth-10 phosphate. Of particular use in the present invention is the Apifil PEG-8 beeswax emulsifier, which is a combination of fatty acid esters and polyethylene glycol and is a nonionic self-emulsifying base.

Another usable class of emulsifiers comprises dimethicone copolymers, for example polyoxyalkylene modified dimethylpolysiloxanes. The polyoxyalkylene group may be polyoxyethylene (POE) or polyoxypropylene (POP) or a copolymer of POE and POP in these silicone derivatives. Other useful silicone derivatives include Silshine® 151 (phenylpropyldimethylsiloxysilicate), SM-2115® (amodimethicone/nonionic emulsion), SM-2125® (amodimethicone/cationic emulsion), SM-2785® (anionic dimethiconol high viscosity emulsion), SME-253® and SR-1000® all of which are available from Momentive Performance Materials (GE Silcones).

Other suitable emulsifiers and are widely available under many trade names and designations including Abil®, Arlacel®, Arlatone®, Brij®, Cremophor®, Dehydrol®, Dehymuls®, Emerest®, Jaguar®, Lameform®, Liponate®, Pluronic®, Prisorine®, Quest PGPR®, Span®, Tween®, SF1228, DC3225C and Q2-5200.

Most preferred emulsifiers for use herein include SM-2115® (amodimethicone/nonionic emulsion from Momentive Performance Materials), Polysorbate-20 (e.g. Tween® 20 from Uniqema), PEG-5 to PEG-40 Hydrogenated Castor Oil (e.g. various Arlatone® from Croda or Lumulse® emulsifiers from Lambent), propylene glycol dicaprylate/dicaprate (e.g. Liponate® PC from Lipo), polyoxyethylene fatty alkyl mixed glyceride (e.g. PEG-6 caprylic/capric glyceride sold as Glycerox® 767 from Croda), or mixtures of any combinations of these, at levels of from about 0.01% to about 30% by weight in the total composition.

The compositions of the present invention may also include conditioners. Most preferred are the cationic conditioners. Non-limiting examples of such include the fatty alkyl quaternary salts such as stearalkonium chloride (available from Croda under the trade name Incroquat® SDQ-25), minkamidopropyl dimethyl 2-hydroxyethyl ammonium chloride (Incroquat® 26 from Croda), 18-methyleicosanoic acid fatty quat (Incroquat® Behenyl 178-MEA® from Croda), various ester and diester quaternary substances, imidazoline quaternaries, behenyl trimethyl ammonium chloride (for example INCROQUAT® TMC-80 from Croda and ECONOL® TM22 from Sanyo Kasei), distearyl dimethyl ammonium chloride (for example Varisoft® TA 100 from Goldschmidt), cetyl trimethyl ammonium chloride (for example CA-2350® from Nikko Chemicals), hydrogenated tallow alkyl trimethyl ammonium chloride, dialkyl (C₁₄-C₁₈) dimethyl ammonium chloride, ditallow alkyl dimethyl ammonium chloride, dihydrogenated tallow alkyl dimethyl ammonium chloride, dicetyl dimethyl ammonium chloride, di(behenyl/arachidyl) dimethyl ammonium chloride, dibehenyl dimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate) ammonium chloride, N-(stearoyl colamino formyl methy) pyridinium chloride, and distearoylethyl hydroxyethylmonium methosulfate (e.g., Dehyquart® F75 from Cognis).

Additional cationic conditioning materials that may find use in the present invention include the cationic guar derivatives, most notably the products from Rhodia under the Jaguar® trade name. Although mentioned above in the context of emulsifiers/thickeners (most useful are the nonionic guar materials for that purpose), the cationic guar materials are most appropriate as conditioners. One notable example includes guar hydroxypropyltrimonium chloride (available from Rhodia in various grades and forms under the trade names; Jaguar® C-13-S, Jaguar® C-14-S, Jaguar® C-162, Jaguar® C-17, Jaguar® C-1000, and Jaguar® EXCEL®).

Other cationic materials may find use in the present invention. Such cationics are quaternary ammonium compounds having at least one group selected from the group consisting of an ester group, an amido group, and mixtures thereof. Non-limiting examples of such cationics include di-(alkylcarboxyethyl) hydroxyethylmethylammonium methosulfate (for example Rewoquat® V3620 available from Goldschmidt), and methyl bis-(alkylamidoethyl) 2-hydroxyethylammonium methosulfate (for example Varisoft® 222 LT-90 available from Goldschmidt).

Salts of amidoamines and acids can be used as cationics for the present compositions. The amidoamine useful herein are those having the following general formula: R¹CONH(CH₂)ₘN(R²)₂ wherein R¹ is a residue of C₁₁ to C₂₄ fatty acids, R² is a C₁ to C₄ alkyl, and m is an integer from 1 to 4. Preferred amidoamines useful in the present invention includes stearamidopropyidimethylamine, stearamidopropyldiethylamine, stearamidoethyl diethyl amine, stearamido ethyl dimethylamine, palmitamidopropyl dimethylamine, palmitamidopropyl diethylamine, palmitamidoethyl diethylamine, palmitamidoethyl dimethylamine, behenamidopropyl dimethylamine, behenamidopropyl idiethylamine, behenamidoethyl diethylamine, behenamidoethyl dimethylamine, arachidamidopropyl dimethylamine, arachidamidopropyl diethylamine, arachidamidoethyl diethylamine, arachidamidoethyl dimethylamine, and mixtures thereof; more preferably stearamidopropyl dimethylamine, stearamidoethyl diethylamine, and mixtures thereof. Commercially available amidoamines useful herein include: stearamidopropyl dimethylamine having tradename SAPDMA® available from Inolex, and tradename Amidoamine® MPS available from Nikko.

Most preferred cationic conditioners for use in the present invention include stearalkonium chloride (Incroquat® SDQ-25 from Croda), guar hydroxypropyltrimonium chloride (Jaguar® C-14-S and other grades/forms from Rhodia), or both combined, at a level of from about 0.1% to about 5% by weight total cationic conditioner in the composition.

The compositions of the present invention may also contain a humectant. As mentioned previously, polyols can function as humectants (simple diols, sugar alcohols and even polymeric glycols), but other materials may be added in conjunction with polyols, or on their own as humectants for use in the present compositions. Many of these preferred humectants will have one or more hydroxyl groups attached, non limiting examples of which include liquid polyalkylene glycols such as polypropylene glycols and diethylene or polyethylene glycols (e.g., molecular weights from about 200 to about 600, such as PEG-4, PEG-6, PEG-8, and PEG-12) ethyl hexanediol, hexylene glycol, butylene glycol, glycerin, propylene glycol, dipropylene glycol, tripropylene glycol, polypropylene-5-laureth-5, polyglycerol cocoate, sorbitol, fructose, glycine, inositol, panthenol and combinations thereof, some of which were mentioned above since they may have multiple function within these compositions.

Other non-limiting examples of suitable humectants include Aloe Vera, Lubrajel Oil® (glyceryl polymethacrylate and propylene glycol), Glucquat® 125 (lauryl methyl gluceth-10 hydroxypropyldimonium chloride), Glucam® E-10 (methyl gluceth-10), Glucam® E-20 (methyl gluceth-20), Glucam® P-10 (PPG-10 methyl glucose ether), Glucam® P-20 (PPG-20 methyl glucose ether), sodium lactate, sodium PCA, Schercomid® LME (Lactamide MEA), Clearcol® (soluble collagen), Collasol® M (soluble collagen), Crolastin® (hydrolyzed elastin), Cromoist® CS (sodium chrondroitin sulfate and hydrolyzed collagen), Cromoist® HYA (hydrolyzed collagen and hyaluronic acid), Cromoist® WHYA (hydrolyzed wheat protein and hylauronic acid), Cromoist® O-25 (hydrolyzed oats), Cropeptide® W (hydrolyzed wheat protein and hydrolyzed wheat starch), Crosilk® 10,000 (hydrolyzed silk), Crosilk® Liquid (silk amino acids), Crosilkquat® (cocodimoniumhydroxypropyl silk amino acids), Crotein® CAA/SF (collagen amino acids), Crotein® HKP (hair keatin amino acids and sodium chloride, Crotein® HKP/SF (keratin amino acids), Crotein® MCAA (collagen amino acids), Hydrolactin® 2500 (hydrolyzed milk protein), Hydrosoy® 2000 (hydrolyzed soy protein), Hydrotriticum® 2000 (hydrolyzed wheat protein), Hydrotriticum® WAA (wheat amino acids), Reticusol (hydrolyzed reticulin), Tritisol® (hydrolyzed wheat protein), Incromectant® AMEA-100 (acetamide MEA), Incromectant® AMEA-70 (acetamide MEA), Incromectant® LMEA (acetamide MEA and lactamide MEA), Incromectant® AQ (acetamidopropyltrimonium chloride), Incromectant® LQ (lactamidopropyl trimonium chloride), Gelatin NF®, Lactil® (sodium lactate, sodium PCA, glycine, fructose, urea, niacinamide, inocitol, sodium benzoate and lactic acid).

The preferred humectant for use herein is Glucam® E-10 (methyl gluceth-10 available from Noveon) and/or panthenol at from about 0.01% to about 1% by weight in the composition.

The compositions of the present invention may also include a high molecular weight water-soluble poly(ethylene oxide) polymeric resin. These materials are available from Dow Chemical Co. under the trade name Polyox® WSR (for “water soluble resin”). For example, of particular use in the present compositions are Polyox® WSR-N-3000, Polyox® WSR-205, Polyox® WSR-1105, Polyox® WSR-N-12K, Polyox® WSR-N-60K, Polyox® WSR-301, Polyox® WSR-COAGULANT, Polyox® WSR-303, and Polyox® WSR-308. Most preferred for use herein is Polyox® WSR-301, (4,000,000 approx. molecular weight poly(ethylene oxide) polymer from Dow Chemical) at from about 0.001% to about 1% by weight in the composition.

The compositions of the present invention may also contain an emulsifying wax and/or oil. Such materials include the non-limiting examples of bees' wax, candelilla wax, carnauba wax, emulsifying wax (for example Polawax® from Croda) and Jojoba, safflower, canola (tribehenin), tallow, lard, palm, castor, sunflower seed, or soya bean oil oils, or hydrogenated derivatives thereof. Most preferred is to incorporate Polawax®, jojoba oil, safflower oil, tribehenin, and/or hydrogenated castor oil, singularly or in any combination. When desired in the present compositions, any combination of these materials may be used at from about 0.1% to about 5% by weight of the total composition.

The compositions of the present invention may also contain solvent, for example to adjust physical parameters, or give consumer benefits, or to adapt the base formulation to a specific product physical form and/or delivery system (liquid, gel, pump, aerosol, etc.). For example, it may be necessary to add small amounts of various solvents to the composition to help optimize viscosity, stability, feel, dry time, droplet size when sprayed, etc., or even to add greater amounts to convert the base formulas that may be gels or thick emulsions into thin liquids amenable to aerosol hair sprays or non-aerosol pump sprays. In addition to adding the required constituent water, (the primary solvent for the compositions of the present invention), various alkanol solvents may be added as well. Examples of such solvents include but are not limited to ethanol, propanol, isopropanol, n-butanol, iso-butanol, tert-butanol and the like, or monoalkyl and/or dialkyl ether solvents including materials such as propylene glycol monomethyl ether, propylene glycol monobutyl ether, propylene glycol monophenyl ether, propylene glycol monohexyl ether, ethyleneglycol monomethyl ether, ethylene glycol monobutyl ether, ethylene glycol monophenyl ether, ethylene glycol monohexyl ether, dipropylene glycol monomethyl ether, dipropylene glycol monobutyl ether, dipropylene glycol monophenyl ether, dipropylene glycol monohexyl ether, diethylene glycol monobutyl ether (butyl carbitol), triethylene glycol monobutyl ether, mono, di, tri propylene glycol monobutyl ether, tetraethylene glycol monobutyl ether, mono, di, tripropylene glycol monomethyl ether, propylene glycol monomethyl ether, ethylene glycol monohexyl ether, diethylene glycol monohexyl ether, propylene glycol tertiary butyl ether, ethylene glycol monoethyl ether, ethylene glycol monomethyl ether, ethylene glycol monopropyl ether, ethylene glycol monopentyl ether, diethylene glycol monomethyl ether, diethylene glycol monoethyl ether, diethylene glycol monopropyl ether, diethylene glycol monopentyl ether, triethylene glycol monomethyl ether, triethylene glycol monoethyl ether, triethylene glycol monopropyl ether, triethylene glycol monopentyl ether, triethylene glycol monohexyl ether, mono, di, tripropylene glycol monoethyl ether, mono, di tripropylene glycol monopropyl ether, mono, di, tripropylene glycol monopentyl ether, mono, di, tripropylene glycol monohexyl ether, mono, di, tributylene glycol mono methyl ether, mono, di, tributylene glycol monoethyl ether, mono, di, tributylene glycol monopropyl ether, mono, di, tributylene glycol monobutyl ether, mono, di, tributylene glycol monopentyl ether and mono, di, tributylene glycol monohexyl ether, ethylene glycol monoacetate and dipropylene glycol propionate and the like. Many of these types of solvents are available from Dow Chemical Co. under the DOWANOL® brand. Additionally, solvents such as acetone, methyl acetate, d-limonene and the like may find use in the present invention. The solvents may be present from about 1% to about 50% by weight of the composition. Most preferred for use here is ethanol and/or ethylene glycol monophenyl ether (phenoxyethanol) at from about 1% to about 50% by weight in the composition.

The water in the compositions of the present invention may be present at levels from about 1% to about 90%

The compositions of the present invention may also include protein, natural or a derivatized for hair strengthening purposes. For example, hydrolyzed vegetable protein PG-propyl silanetriol (available from Croda under the trade name Keravis®), or hydrolyzed jojoba protein (available from the Desert Whale Jojoba Co. under the trade name Jojoba Pro-HP®) are preferred for incorporation in the present invention. When desired, these protein products may be used individually or combined at from about 0.01% to about 5% by weight of the composition.

Additionally, it may be useful to adjust the final pH of the hair styling compositions. That being said, a pH of from about 5 to about 7.5 is preferred and, depending on the other adjuvant added to the composition (e.g., acrylic rheology modifiers that require neutralization with alkali in order to thicken), adjustment of pH “up” or “down” might be necessary. To that end, various acids or alkalis may be added, including but not limited to, mineral acids, fatty acids, and organic acids, and ammonia, various alkylamines, alkanolamines and hydroxides. Most preferred is to add citric acid and/or aminomethylpropanol (AMP) to adjust pH to within the preferred range mentioned. These preferred buffering agents might also serve other purposes as mentioned previously, (e.g., chelating of minerals or corrosion inhibition in metal aerosol cans or metal squeeze tubes, described below).

For aerosol preparations, the compositions may include one or more gaseous propellants and various corrosion inhibitors that are typically included when aerosols are packaged in steel or aluminum cans. For example, the base compositions described (i.e., liquid composition) may be aerosolized with hydrocarbons, ethers, CO₂, nitrogen, compressed air or fluorinated hydrocarbon gasses. Most preferred is to aerosolize the compositions of the present invention with dimethylether (DME) or to use a bag-in-can assembly and pressurize the space between the bag and can with compressed air. Depending on the liquid composition, the type of metal can, and the propellant system, various corrosion inhibitors may become necessary. Some non-limiting examples of corrosions inhibitors that may be used are amines, amides, alkanolamines, phosphates, benzoates, and imidazolines. Most preferred is to use aminomethylpropanol such as AMP-95® available from Angus Chemical although it is well known in the aerosol industry that the choice of corrosion inhibitor combinations for aerosols is entirely empirical, requiring trial and error and long storage stability experiments. Other metal packaging that is non-pressurized may also require corrosion inhibitors. For example, metal squeeze tubes for dispensing gel and paste compositions may require these same corrosion inhibitors.

Also optional to the compositions of the present invention is the addition of a chelant. Chelants that may find use herein include but are not limited to citric acid and its various salts, phosphates (organic and inorganic), nitrilotriacetate NTA and associated salts, the various ethylenediaminetetraacetic acid (EDTA) salts, most notably disodium-EDTA (e.g. Hampene® NA2 from Dow Chemical), and lower molecular weight polyacrylates (MW from about 1,000 to about 5,000). For example, the present invention may include disodium- trisodium- or tetrasodium-EDTA, various salts of NTA, phosphate esters, Acusol® 445 from Rohm and Haas (4,500 molecular weight polyacrylate), ascorbic acid, citric acid, monosodium-, disodium- or trisodium citrate. Some of these materials aid stability and, if the final compositions are aerosolized, can help with corrosion inhibition in metal cans. Most preferred for use here are ascorbic acid, citric acid or citrates and/or disodium-EDTA, individually or in total at from about 0.001% to about 1% by weight.

The compositions of the present invention may also include a preservative (i.e., an antimicrobial). Examples of antimicrobial agents that find use in the present invention include glutaraldehyde, formaldehyde, 2-bromo-2-nitropropane-1,3-diol sold under the trade name Bronopol®, 5-chloro-2-methyl-4-isothiazoline-3-one and 2-methyl-4-isothiazoline-3-one sold under the trade name Kathon® from Rohm and Haas, parabens (e.g., methyl-, ethyl-, propyl-, butyl-paraben), methylisothiazolinone sold under the trade name Neolone® from Rohm and Haas, and mixtures thereof. The preferred level for the antimicrobial is from about 0.001% to about 0.1%, or at that level recommended by the supplier of the particular antimicrobial and/or suggested in the supplier technical literature as that level required for optimally preserving hair-care compositions from mold and bacterial growth. The preferred antimicrobials for use in the present invention include the parabens.

Fragrance is an optional ingredient for the hair styling compositions of the present invention. For consumer acceptance, product recognition and repurchase and to help keep hair fresh smelling, a fragrance is preferably added to the hair-hold compositions of the present invention. The fragrance in the composition of the present invention may comprise one of more volatile organic compounds available from any of the now known, or hereafter established, perfumery suppliers, such as International Flavors and Fragrances (IFF) of New Jersey, Givaudan of New Jersey, Firmenich of New Jersey, etc. Many types of fragrances can be used in the present invention. Preferably the fragrance materials are volatile essential oils. The fragrances, however, may be synthetically derived materials (aldehydes, ketones, esters, etc.), naturally derived oils, or mixtures thereof. Naturally derived fragrance substances include, but are not limited to, musk, civet, ambergis, castoreum and like animal perfumes; abies oil, ajowan oil, almond oil, ambrette seed absolute, angelic root oil, anise oil, basil oil, bay oil, benzoin resinoid, bergamot oil, birch oil, bois de rose oil, broom abs., cajeput oil, cananga oil, capsicum oil, caraway oil, cardamon oil, carrot seed oil, cassia oil, cedar leaf, cedarwood oil, celery seed oil, cinnamon bark oil, citronella oil, clary sage oil, clove oil, cognac oil, coriander oil, cubeb oil, cumin oil, camphor oil, dill oil, estragon oil, eucalyptus oil, fennel sweet oil, galbanum res., garlic oil, geranium oil, ginger oil, grapefruit oil, hop oil, hyacinth abs., jasmin abs., juniper berry oil, labdanum res., lavander oil, laurel leaf oil, lavender oil, lemon oil, lemongrass oil, lime oil, lovage oil, mace oil, mandarin oil, mimosa abs., myrrh abs., mustard oil, narcissus abs., neroli bigarade oil, nutmeg oil, oakmoss abs., olibanum res., onion oil, opoponax res., orange oil, orange flower oil, origanum, orris concrete, pepper oil, peppermint oil, peru balsam, petitgrain oil, pine needle oil, rose abs., rose oil, rosemary oil, sandalwood oil, sage oil, spearmint oil, styrax oil, thyme oil, tolu balsam, tonka beans abs., tuberose abs., turpentine oil, vanilla beans abs., vetiver oil, violet leaf abs., ylang ylang oil and like vegetable oils, etc. Synthetic fragrance materials include but are not limited to pinene, limonene and like hydrocarbons; 3,3,5-trimethylcyclohexanol, linalool, geraniol, nerol, citronellol, menthol, borneol, borneyl methoxy cyclohexanol, benzyl alcohol, anise alcohol, cinnamyl alcohol, β-phenyl ethyl alcohol, cis-3-hexenol, terpineol and like alcohols; anethole, musk xylol, isoeugenol, methyl eugenol and like phenols; α-amylcinnamic aldehyde, anisaldehyde, n-butyl aldehyde, cumin aldehyde, cyclamen aldehyde, decanal, isobutyl aldehyde, hexyl aldehyde, heptyl aldehyde, n-nonyl aldehyde, nonadienol, citral, citronellal, hydroxycitronellal, benzaldehyde, methyl nonyl acetaldehyde, cinnamic aldehyde, dodecanol, α-hyxylcinnamic aldehyde, undecenal, heliotropin, vanillin, ethyl vanillin and like aldehydes; methyl amyl ketone, methyl β-naphthyl ketone, methyl nonyl ketone, musk ketone, diacetyl, acetyl propionyl, acetyl butyryl, carvone, menthone, camphor, acetophenone, p-methyl acetophenone, ionone, methyl ionone and like ketones; amyl butyrolactone, diphenyl oxide, methyl phenyl glycidate, .gamma.-nonyl lactone, coumarin, cineole, ethyl methyl phenyl glicydate and like lactones or oxides; methyl formate, isopropyl formate, linalyl formate, ethyl acetate, octyl acetate, methyl acetate, benzyl acetate, cinnamyl acetate, butyl propionate, isoamyl acetate, isopropyl isobutyrate, geranyl isovalerate, allyl capronate, butyl heptylate, octyl caprylate octyl, methyl heptynecarboxylate, methine octynecarboxylate, isoacyl caprylate, methyl laurate, ethyl myristate, methyl myristate, ethyl benzoate, benzyl benzoate, methylcarbinylphenyl acetate, isobutyl phenylacetate, methyl cinnamate, cinnamyl cinnamate, methyl salicylate, ethyl anisate, methyl anthranilate, ethyl pyruvate, ethyl α-butyl butylate, benzyl propionate, butyl acetate, butyl butyrate, p-tert-butylcyclohexyl acetate, cedryl acetate, citronellyl acetate, citronellyl formate, p-cresyl acetate, ethyl butyrate, ethyl caproate, ethyl cinnamate, ethyl phenylacetate, ethylene brassylate, geranyl acetate, geranyl formate, isoamyl salicylate, isoamyl isovalerate, isobornyl acetate, linalyl acetate, methyl anthranilate, methyl dihydrojasmonate, nopyl acetate, β-phenylethyl acetate, trichloromethylphenyl carbinyl acetate, terpinyl acetate, vetiveryl acetate and like esters, and the like. Suitable fragrance mixtures may produce a number of overall fragrance type perceptions including but not limited to, fruity, musk, floral, herbaceous (including mint), and woody, or perceptions that are in-between (fruity-floral for example). Typically these fragrance mixtures are compounded by mixing a variety of these active fragrance materials along with various solvents to adjust cost, hedonics and intensity of perception within the final product composition. Thus a preferred fragrance for use in the compositions of the present invention may be comprised of a mixture of many fragrance actives and volatile solvents, sometimes along with smaller amounts of emulsifiers, stabilizers, wetting agents and preservatives. More often than not, the compositions of the fragrance mixtures purchasable from the various fragrance supply houses remain proprietary. The fragrance is preferably incorporated at a level of from about 0.01% to about 2% by weight, or at that level where consumer acceptance is maximized.

Dyes are optional ingredients within the compositions of the present invention. Dyes may comprise pigments, or other colorants, chosen so that they are compatible with the composition and lend consumer acceptability of the final product. Any of the FD&C colorants may be used along with permanent dyes and pigments, anti-graying ingredients, and glitter or other suspended “sparkle”. Non-limiting examples of colorants for use in the present invention is Liquitint® Green FS (from Milliken), at from about 0.001% to about 0.01% by weight, based on the entire composition. Other dyes such as C.I. Pigment Green #7, C.I. Reactive Green #12, F D & C Green #3, C.I. Acid Blue #80, C.I. Acid Yellow #17, Liquitint® Red MX, F D & C Yellow #5, Liquitint® Violet LS, Fast Turquise GLL, Liquitint® Blue MC, or mixtures thereof are also useful in the compositions of the present invention.

Exemplary embodiments of hair-hold compositions of the present invention are shown below in Table 1. It is important to note that the formulas presented in the table represent the “liquid phase” of the composition. If desired, these liquid formulations may be adapted to aerosol products by addition of propellant and adjustment of solvents.

TABLE 1

| Ingredients | Formula 1 | Formula 2 | Formula 3 | Formula 4 | Formula 5 |
|---|---|---|---|---|---|
| Disaccharide (Maltose, Sucrose and the like) | 15.000 | 15.000 | | | |
| Monosaccharide (Fructose, Glucose and the like) | | | 4.000 | 15.000 | 5.000 |
| Starch and Cellulose Materials | | | | | |
| Hydroxypropyl Starch Phosphate, Cornstarch- | 6.000 | 1.125 | 0.800 | 0 | 0 |
| modified, Hydroxy ethyl cellulose, and the like. | | | | | |
| Polyols, fatty alcohols, solvents, emulsifiers, oils and waxes | | | | | |
| PEG-90, Propylene Glycol, Panthenol, Glycerol, | 1.920 | 1.900 | 6.950 | 1.900 | 36.000 |
| Cetyl Alcohol Ethylene glycol monophenyl ether, | | | | | |
| Polysorbate-20, Methyl Gluceth-10, Emulsifying | | | | | |
| Wax, safflower oil, Jojoba oil, and the like. | | | | | |
| Acrylates and/or silicone-polymer thickeners | | | | | |
| Carbopol Ultrez, Dimethicone copolymers, and the | 0 | 0.250 | 0.400 | 0.500 | 0 |
| like. | | | | | |
| Cationic conditioners | | | | | |
| Stearalkonium chloride, guar hydroxypropyl | 0 | 0 | 2.400 | 0 | 2.600 |
| trimonium chloride, and the like. | | | | | |
| Proteins | | | | | |
| Hydrolyzed proteins (Keravis, Jojoba Pro-HP, etc.) | 0 | 0 | 3.000 | 0 | 0 |
| pH adjusting agents | | | | | |
| EDTA, EDTA-salts, amines, alkanolamines, | 0 | 0.425 | 0.090 | 0.450 | 0.090 |
| organic acids, mineral acids, hydroxides, and the | | | | | |
| like. | | | | | |
| Preservatives, fragrances, and dyes | | | | | |
| Preservatives, fragrances, dyes, and the like. | 0.300 | 0.300 | 0.500 | 0.300 | 1.200 |
| Remainder and total | | | | | |
| Water | 76.780 | 81.000 | 81.860 | 81.850 | 55.110 |
| Total | 100.00 | 100.00 | 100.00 | 100.00 | 100.00 |

PHYSICAL PROPERTIES

| | | | | | |
|---|---|---|---|---|---|
| PH | 5.7 | 6.3 | 5.3 | 6.8 | — |
| Viscosity (#6 @10 rpm) | 26,000 | 20,000 | 40,000 | 16,800 | Paste |

Tables 2-6 below summarize various consumer tests wherein each experimental composition Example 1-3 according to Table 1 was tested against one or more retail products of the same form. Each attribute (shine, hold, volume and flakiness) was ranked on a scale from 1-10, with 1=the worse score and 10=the best score. These consumer tests included a total of four retail market products that are readily available for purchase in stores, and were blindly labeled as PROD-A (a “weightless gel”) and PROD-B (a “strong hold gel”), PROD-C (a “studio melting gel”), PROD-D (a popular styling “potion”), and PROD-E (a retail paste product).

TABLE 2
Example #1 versus two retail “gels”

| | SHINE | HOLD | VOLUME | FLAKINESS |
|---|---|---|---|---|
| Example 1 | 9 | 9 | 9 | 8 |
| PROD-A GEL | 7 | 7 | 7 | 6 |
| PROD-B GEL | 7 | 6 | 6 | 6 |

TABLE 3
Example #2 versus a retail “melting gel”

| | SHINE | HOLD | VOLUME | FLAKINESS |
|---|---|---|---|---|
| Example 2 | 9 | 9 | 9 | 9 |
| PROD-C | 5 | 6 | 5 | 9 |

TABLE 4
Example #3 versus a styling potion

| | SHINE | HOLD | VOLUME | FLAKINESS |
|---|---|---|---|---|
| Example 3 | 9 | 9 | 9 | 9 |
| PROD-D | 6 | 7 | 6 | 9 |

TABLE 5
Example #4 versus a retail “melting gel”

| | SHINE | HOLD | VOLUME | FLAKINESS |
|---|---|---|---|---|
| Example 4 | 9 | 9 | 9 | 9 |
| PROD-C | 5 | 6 | 5 | 9 |

TABLE 6
Example #5 versus a retail “paste”

| | SHINE | HOLD | VOLUME | FLAKINESS |
|---|---|---|---|---|
| Example 5 | 9 | 9 | 9 | 9 |
| PROD-E | 8 | 6 | 6 | 9 |

In summary, I have invented unique hair styling compositions that feature complete replacement of the synthetic fixative polymers with mono- and/or disaccharides to achieve hard-hold characteristics without undue brittleness and flaking. In this way, the formulation of “natural” hair care products is shown to be possible.

I claim:

1. A hair styling composition comprising;
a. at least one monosaccharide or disaccharide;
b. at least one starch, modified starch, cellulose, or modified cellulose selected from the group consisting of hydroxypropyl starch phosphate, methyl cellulose, carboxymethyl cellulose, hydroxypropylmethyl cellulose, hydroxyethyl cellulose, hydroxypropyl cellulose, ethyl hydroxyethyl cellulose, cocodimonium hydroxypropyl oxyethyl cellulose, corn starch, potato starch, oxidized corn starch, hydroxyethyl starch, hydroxypropyl (modified) cornstarch, laurdimonium hydroxypropyl oxyethyl cellulose, nonoxylnyl hydroxyethyl cellulose, steardimonium hydroxyethyl cellulose, tapioca starch, welan, levan, scleroglucan, stachyose, succinoglycan, and wheat starch, and mixtures thereof; and,
c. water,
wherein the composition is devoid of synthetic fixative polymers or copolymers.

2. The hair styling composition of claim 1 further comprising a polyacrylate homopolymer.

3. The hair styling composition of claim 1 further including a diol or polyol.

4. The hair styling composition of claim 1 further including a fatty alcohol.

5. The hair styling composition of claim 1 further including a humectant.

6. The hair styling composition of claim 1 further including a cationic conditioner.

7. The hair styling composition of claim 1 further including a dimethicone polymer.

8. The hair styling composition of claim 1 further including a hydrolyzed protein.

9. The hair styling composition of claim 1 further including a wax or oil chosen from the group consisting of bees' wax, candelilla wax, carnauba wax, emulsifying wax, Jojoba oil, safflower oil, canola oil, tallow, lard, palm oil, castor oil, sunflower seed oil, and soya bean oil, and mixtures thereof.

10. The hair styling composition of claim 1 further including an aerosol propellant, a solvent, and a corrosion inhibitor.

11. The hair styling composition of claim 5, wherein said humectant is ethoxylated methyl glucose ether.

12. The hair styling composition of claim 3, wherein said diol or polyol is chosen from the group consisting of glycerol, polyethylene glycol, and propylene glycol, and mixtures thereof.

13. The hair styling composition of claim 12, wherein the said monosaccharide or disaccharide is chosen from the group consisting of maltose, fructose, and sucrose, and mixtures thereof.

14. The hair styling composition of claim 13, wherein said monosaccharide or disaccharide is incorporated at a level of from about 1% to about 30% by weight in the composition.

15. The hair styling composition of claim 1 further comprising adjuvant selected from the group consisting of fragrances, dyes, pigments, pH adjusting agents, and chelants, and mixtures thereof.

16. A hair styling composition comprising;
a. at least one monosaccharide or disaccharide chosen from the group consisting of maltose, fructose, and sucrose, and mixtures thereof;
b. at least one starch, modified starch, cellulose, or modified cellulose selected from the group consisting of hydroxypropyl starch phosphate, methyl cellulose, carboxymethyl cellulose, hydroxypropylmethyl cellulose, hydroxyethyl cellulose, hydroxypropyl cellulose, ethyl hydroxyethyl cellulose, corn starch, potato starch, oxidized corn starch, hydroxyethyl starch, hydroxypropyl (modified) cornstarch, tapioca starch, and wheat starch, and mixtures thereof;
c. a diol or polyol;
d. a cationic conditioner;
e. a fatty alcohol; and,
f. water,
wherein the composition is devoid of synthetic fixative polymers or copolymers.

17. The hair styling composition of claim 16, wherein said diol or polyol is chosen from the group consisting of glycerol, propylene glycol, and polyethylene glycol, and mixtures thereof.

18. The hair styling composition of claim 16 further including a humectant.

19. The hair styling composition of claim 16 further including a wax or oil chosen from the group consisting of bees' wax, candelilla wax, carnauba wax, emulsifying wax, Jojoba oil, safflower oil, canola oil, tallow, lard, palm oil, castor oil, sunflower seed oil, and soya bean oil, and mixtures thereof.

20. The hair styling composition of claim 16 further comprising adjuvant selected from the group consisting of fragrances, dyes, pigments, pH adjusting agents, and chelants, and mixtures thereof
